(19) United States
(12) Patent Application Publication
(10) Pub. No.: US 2009/0104635 A1
(43) Pub. Date: Apr. 23, 2009

(54) Fluorescent Dry Test Strip Biosensor

(76) Inventor: Tom Cheng Xu, Castro Valley, CA (US)

Correspondence Address: Ashok Tankha, 36 Greenleigh Drive, Sewell, NJ, 08080, US

(21) Appl. No.: 12/207,499

(22) Filed: Sep. 10, 2008

Related U.S. Application Data
(60) Provisional application No. 60/999,547, filed on Oct. 19, 2007.

(51) Int. Cl. C12Q 1/62; C12M 1/00; C12Q 1/00; C12Q 1/54; C12N 11/00; C12Q 1/48; C12Q 1/60; C12Q 1/28

(52) U.S. Cl. 435/10; 435/287.8; 435/4; 435/28; 435/11; 435/14; 435/15; 435/174

(57) ABSTRACT

Disclosed herein is a dry fluorescence biosensor strip for rapid detection of a target analyte present in bodily fluids. The dry fluorescence biosensor strip comprises a sample receptacle and a dry detection membrane. The sample receptacle receives a sample of one of the bodily fluids. The dry detection membrane detects presence of the target analyte in the received sample based on fluorescence induced on the dry detection membrane. Fluorescent signals are emitted from the dry detection membrane on induction of fluorescence. A fluorometer quantifies measurable properties of the target analyte based on the emitted fluorescent signals. The dry fluorescence biosensor strip may further comprise a filtration membrane for filtering the received sample. The filtered sample migrates from the filtration membrane to the dry detection membrane. The dry detection membrane may then detect presence of the target analyte in the filtered sample.

### CROSS REFERENCE TO RELATED APPLICATIONS

This application claims the benefit of provisional patent application No. U.S. 60/999547 titled “Fluorescent dry strip biosensors”, filed on Oct. 19, 2007 in the United States Patent and Trademark Office.

### BACKGROUND

This invention, in general, relates to test strip biosensors. More particularly, this invention relates to a dry fluorescence biosensor strip for rapid detection of an analyte and quantification of measurable properties of an analyte in bodily fluids.

Self monitoring of biological components from human body fluids is required for controlling disease conditions and maintaining a normal life for some individuals. For example, patients with diabetes may need to test blood sugar levels periodically to keep track of the patients' diet, exercises required, and medical treatment. The measured blood sugar levels give an informative feedback to the patients regarding changes required in the patients' eating habits, exercises performed, and intake of medicines for normal daily life. Typically, blood sugar levels are monitored using commercially available dry test strips with a hand held test system.

High cholesterol level in blood may be an indicator for risk of a coronary heart disease in patients. Managing the cholesterol level in the blood may be essential for a healthy heart. Self monitoring of the cholesterol level in the blood may also be performed by patients using the commercially available dry test strips with the hand held test system. The biological components in the bodily fluids such as enzymes also need to be tested. For example, alanine aminotransferase (ALT) is an enzyme that catalyzes the conversion of alanine to pyruvate. Aspartic acid aminotransferase (AST) is an enzyme that catalyzes the conversion of aspartic acid to oxaloacetate. Both ALT and AST enzymes are found in the liver. ALT levels and AST levels in the bodily fluids are a reflection of alterations in liver function.

Self monitoring of the AST levels and ALT levels in the bodily fluids may be needed to ascertain the measure of damage to the liver. The liver may be damaged due to infectious diseases such as hepatitis A, B, or C. In blood banks, the donated blood is screen tested for hepatitis. The screen testing of the donated blood may be performed by measuring the ALT levels and the AST levels. The damage to the liver may also be due to excessive intake of alcohol or drugs. Some cholesterol or lipid-lowering drugs may cause a persistent increase in ALT levels and AST levels in blood serum. Patients consuming the cholesterol or lipid-lowering drugs should be tested for the ALT and the AST enzymes periodically to monitor liver conditions of the patients. Therefore, there is a need for a rapid and accurate test method for detecting ALT and AST enzymes.

The blood sugar levels or the cholesterol level in a sample of the bodily fluids may be measured without difficulty using conventional hand held test systems. Detecting and quantifying the ALT levels and AST levels in the bodily fluids may require elaborate bodily fluid sample preparation and an analytical testing process. The conventional hand held test systems may have bulky structural designs for accommodating testing equipment. The measurement of the biological components in the bodily fluids using the conventional hand held test systems may not be accurate. In order to make the hand held test system compact and portable, an analyte sensitive detection technique and a simplified test strip design may be required.

The analyte sensitive detection technique may be based on fluorescence. Fluorescence is an optical phenomenon in cold bodies characterized by emission of light from the cold bodies on absorption of external radiation. Application of the analyte sensitive detection technique based on fluorescence may require elements such as an excitation source or light source, a fluorescent or fluorogenic molecule, a wavelength filter to isolate emission photons from excitation photons, and a detector that detects emitted photons to report relative output signal intensity. Configuring the elements for detection of the biological components in the bodily fluids may be required to optimize detection of fluorescence. The analyte sensitive detection technique based on fluorescence may require a combination of hardware and software components and miniaturization of the test strip may be problematic. Moreover, conventional methods take a significant amount of time to detect the target analyte.

Factors critical to the fluorescence detection may comprise background fluorescence, fluorescence quenching, and photobleaching. The fluorescence detection sensitivity may be compromised by the background fluorescence. The background fluorescence may originate from endogenous sample constituents or surroundings of a fluorophore causing the fluorescence. The fluorescence quenching decreases the intensity of fluorescence emission. Solvents used in the detection technique, pH value, and bodily fluid assay conditions may cause the fluorescence quenching and may reduce efficiency of the fluorescence detection. Under high intensity illumination conditions, irreversible destruction or the photobleaching of excited fluorophore may occur and may limit the fluorescence detection. Hence there is a need for a hand held dry fluorescence biosensor strip that enables self monitoring and rapid detection of biological components in the sample of the bodily fluids based on fluorescence. Furthermore, there a need for the analyte sensitive detection technique based on fluorescence to overcome the problems associated with the factors critical to the fluorescence detection.

### SUMMARY OF THE INVENTION

This summary is provided to introduce a selection of concepts in a simplified form that are further described in the detailed description of the invention. This summary is not intended to identify key or essential inventive concepts of the claimed subject matter, nor is it intended for determining the scope of the claimed subject matter.

The dry fluorescence biosensor strip disclosed herein addresses the above stated needs for a hand held dry fluorescence biosensor strip that enables self monitoring of biological components in a sample of the bodily fluids based on fluorescence. The dry fluorescence biosensor strip disclosed herein is used for rapid detection of a target analyte present in the bodily fluids. The dry fluorescence biosensor strip disclosed herein comprises a sample receptacle and a dry detection membrane. The sample receptacle receives the sample of one of the bodily fluids. The bodily fluid sample may be one of blood, blood serum, plasma, saliva, urine, mucous fluid, milk, urea, etc.

The dry detection membrane is impregnated with fluorogenic substrates and enzymes. The fluorogenic substrates may be selected from a group comprising dihydrofluoresceins, dihydrocalcein, dihydrorhodamines, dihydroethidium, and 10-acetyl-3,7-dihydroxyphenoxazine. The enzymes may comprise a fluorescence causing enzyme and analyte specific enzymes. The fluorescence causing enzyme may be horseradish peroxidase. The analyte specific enzymes, for example, pyruvate oxidase, or cholesterol oxidase, may be specific to the target analyte being detected and may oxidize the target analyte. The dry detection membrane may also be impregnated with chemical agents. The chemical agents may comprise, for example, stabilizing agents and conditioning agents. The target analyte may comprise, for example, one of glucose, cholesterol, triglyceride, uric acid, creatine kinase, alanine aminotransferase (ALT), or aspartic acid aminotransferase (AST).

The dry detection membrane receives the bodily fluid sample from the sample receptacle. The dry detection membrane detects presence of the target analyte in the received sample based on fluorescence induced on the dry detection membrane. The dry fluorescence biosensor strip disclosed herein may further comprise a filtration membrane. The filtration membrane may first receive the sample of one of the bodily fluids through the sample receptacle. The filtration membrane may then filter the received sample. For example, the filtration membrane may separate plasma from red blood cells present in a sample of blood. The filtration membrane may also comprise chemical or biological reagents to facilitate the detection of the target analyte. The filtration membrane may be pretreated with the chemical or biological reagents to condition the bodily fluid sample for accurate analyte detection. The filtered sample migrates from the filtration membrane to the dry detection membrane.

If plasma or blood serum is used as the bodily fluid sample then the filtration membrane may not be required and the dry detection membrane may receive the bodily fluid sample of plasma or the blood serum directly from the sample receptacle. The dry detection membrane may then detect the presence of the target analyte in the filtered sample based on fluorescence induced on the dry detection membrane. The dry fluorescence biosensor strip may further comprise an adhesive patch for binding the filtration membrane and the dry detection membrane on a support platform.

The reaction between the target analyte, the fluorogenic substrates, and the enzymes induces fluorescence rapidly on the dry detection membrane. The induction of fluorescence on the dry detection membrane results in emission of fluorescent signals from the dry detection membrane. The dry fluorescence biosensor strip detects the target analytes in about ten seconds from the time of receiving the bodily fluid sample. The emitted fluorescent signals from the dry detection membrane are read and measured by a fluorometer. The fluorometer may be used to quantify the measurable properties of the detected target analyte. For example, the fluorometer may determine a measurable property such as concentration of the detected target analyte in the received sample. The dry fluorescence biosensor strip detects the target analytes in about ten seconds from time of receiving the bodily fluid sample.

### BRIEF DESCRIPTION OF THE DRAWINGS

The foregoing summary, as well as the following detailed description of the invention, is better understood when read in conjunction with the appended drawings. For the purpose of illustrating the invention, exemplary constructions of the invention are shown in the drawings. However, the invention is not limited to the specific methods and instrumentalities disclosed herein.

FIG. 1 illustrates a dry fluorescence biosensor strip for rapid detection of a target analyte present in bodily fluids.

FIG. 2 illustrates an exploded view of a dry fluorescence biosensor strip for rapid detection of a target analyte present in bodily fluids.

FIG. 3 exemplarily illustrates a top view of a dry fluorescence biosensor strip.

FIG. 4 exemplarily illustrates a bottom view of a dry fluorescence biosensor strip.

FIG. 5 exemplarily illustrates a graph correlating ALT concentration with the reaction rate measured by fluorescent intensity for serially diluted ALT standard samples.

FIG. 6 exemplarily illustrates a graph correlating ALT concentration with the reaction rate measured by fluorescent intensity for patient bodily fluid samples.

FIG. 7 exemplarily illustrates a graph correlating cholesterol concentration with the reaction rate measured by fluorescent intensity.

### DETAILED DESCRIPTION OF THE INVENTION

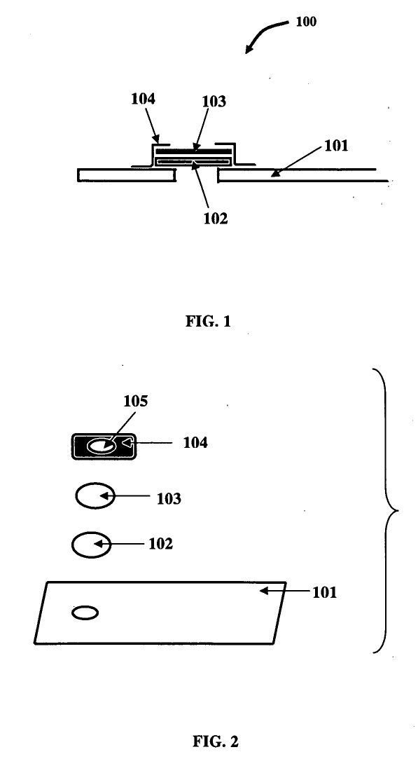

FIG. 1 illustrates a dry fluorescence biosensor strip 100 for rapid detection of a target analyte present in bodily fluids. The dry fluorescence biosensor strip 100 comprises a sample receptacle 105, a dry detection membrane 102, an adhesive patch 104, and a support platform 101. The dry detection membrane 102 is bound to the support platform 101 using the adhesive patch 104. A sample of one of the bodily fluids is introduced into the sample receptacle 105 of the dry fluorescence biosensor strip 100. The sample receptacle 105 receives the sample of one of the bodily fluids. The bodily fluid sample may be, for example, one of blood, blood serum, plasma, saliva, urine, mucous fluid, milk, urea, etc. The sample receptacle 105 may comprise a circular opening for receiving the bodily fluid sample as illustrated in FIG. 2 and FIG. 3. The dry detection membrane 102 receives the bodily fluid sample from the sample receptacle 105. The dry detection membrane 102 detects presence of the target analyte in the received sample.

The dry detection membrane 102 is impregnated with fluorogenic substrates and enzymes. The fluorogenic substrates may be selected from a group comprising dihydrofluoresceins, dihydrocalcein, dihydrorhodamines, dihydroethidium, and 10-acetyl-3,7-dihydroxyphenoxazine (ADHP). The enzymes may comprise a fluorescence causing enzyme and analyte specific enzymes. The fluorescence causing enzyme may be horseradish peroxidase (HRP). The analyte specific enzymes may be specific to the target analyte being detected and may oxidize the target analyte. For example, pyruvate oxidase may be used for detection of presence of ALT in the bodily fluid sample. Cholesterol oxidase may be used for detection of presence of cholesterol in the bodily fluid sample. The dry detection membrane 102 may also be impregnated with chemical agents. The chemical agents may comprise, for example, stabilizing agents and conditioning agents.

The dry detection membrane 102 detects the presence of the target analyte based on fluorescence induced on the dry detection membrane 102. The dry detection membrane 102 detects the presence of alanine aminotransferase (ALT) in the bodily fluids. The dry detection membrane 102 also detects presence of aspartic acid aminotransferase (AST) in the bodily fluids. The dry detection membrane 102 may further detect the presence of target analytes, for example, glucose, cholesterol, triglyceride, uric acid, creatine kinase, etc.

Commercially available membranes may be used for preparing the dry detection membrane 102. For example, nylon biodyne membranes and supor membranes, both from Pall Life Sciences, Inc., may be used as the detection membrane for preparing the dry detection membrane 102. The pore sizes of the detection membrane used to prepare the dry detection membrane 102 may be about 0.2 microns to about 10 microns. A reagent solution may be formulated with buffering reagents, a fluorogenic substrate, the enzymes, the conditioning agents, and the stabilizing agents.

The detection membrane may then be impregnated with the reagent solution. After blotting off extra liquid from surface of the detection membrane, the impregnated detection membrane may be dried in circulating air. The buffering reagents may comprise chemicals, for example, tri-sodium citrate with citric acid and potassium phosphate di-basic with potassium phosphate mono-basic. The pH of the buffering reagents may be about 6 to about 8.

The reaction between the target analyte, the fluorogenic substrates and the enzymes on the dry detection membrane 102 induces the fluorescence rapidly. The induction of the fluorescence results in emission of fluorescent signals from the dry detection membrane 102. For example, the target analyte to be detected may either be oxidized into hydrogen peroxide, or may participate in an oxidation pathway to yield hydrogen peroxide utilizing the analyte specific enzymes. The fluorogenic substrate reactive to HRP and hydrogen peroxide may be used in the reagent solution. The fluorogenic substrates may be oxidized by HRP using hydrogen peroxide as an oxidizing agent to induce the fluorescence on the dry detection membrane 102. For example, ADHP may be used as the fluorogenic substrate in the reagent solution. ADHP is a sensitive and a stable fluorogenic substrate for HRP. ADHP is also an ultra sensitive probe for hydrogen peroxide. In the presence of HRP and hydrogen peroxide, ADHP generates fluorescent resorufin. The fluorescent resorufin may be excited at about 520 nm to about 530 nm and detected at about 580 nm to about 600 nm of the fluorescent resorufin's emitted fluorescence. Air-oxidation of ADHP is minimal. ADHP can detect trace amounts of hydrogen peroxide.

Hydrogen peroxide is produced in many enzymatic redox reactions. Therefore, ADHP may be also be used in coupled enzymatic reactions to detect the activity of oxidases, related enzymes, substrates, or target analytes such as glucose, cholesterol, triglyceride, uric acid, creatine kinase, ALT, and AST. Then HRP may be used as a universal reporting enzyme to convert the fluorogenic substrate to a fluorescent dye using the hydrogen peroxide to emit the fluorescent signals.

The dry fluorescence biosensor strip 100 may further comprise a filtration membrane 103 as illustrated in FIG. 1. An exploded view of the dry fluorescence biosensor strip 100 comprising the sample receptacle 105, the dry detection membrane 102, and the filtration membrane 103 is illustrated in FIG. 2. The adhesive patch 104 binds the filtration membrane 103 and the dry detection membrane 102 on the support platform 101. The adhesive patch 104 may comprise a circular opening used as the sample receptacle 105 as illustrated in FIG. 2 and FIG. 3. The support platform 101 may contain an adhesive layer on interior surface of the support platform 101 to physically attach the dry detection membrane 102 onto the support platform 101 by lamination or a double sided adhesive tape. The support platform 101 may be constructed from mylar, polyethylene terephthalate, or polyester sheet. The dry detection membrane 102 may be circular in shape and adhered to the support platform 101. The filtration membrane 103 may also be in the size and shape of the dry detection membrane 102. The filtration membrane 103 may be laid on top of the dry detection membrane 102. The adhesive patch 104 may then be attached to the support platform 101 to bind the filtration membrane 103 and the dry detection membrane 102 on the support platform 101.

The filtration membrane 103 filters the received sample. For example, the filtration membrane 103 may separate plasma from the red blood cells present in the received sample of blood. Examples of commercially available filtration membranes may be CS and SG membranes from Nanogen Point of Care Diagnostics Division, Toronto, Ontario Canada, and BTS-SP membranes from Pall Life Sciences, Ann Arbor, Mich. The BTS-SP membranes are asymmetric membranes specifically engineered for serum separation of whole blood sample. Graduated pore structure of the asymmetric membranes comprises open pores on upstream side of the asymmetric membranes and finer pores on downstream side of the asymmetric membranes. The asymmetry of the graduated pore structure allows the red blood cells to be captured in the open pores and the plasma wicks into the finer pores on the downstream side of the asymmetric membranes. The upstream side of the asymmetric membranes with open pores is also an absolute cell exclusion zone.

The filtration membrane 103 may also comprise chemical or biological reagents to facilitate the detection of the target analyte. The filtration membrane 103 may be pretreated with the chemical or biological reagents to condition the bodily fluid sample for accurate analyte detection. For example, the filtration membrane 103 may be pretreated with sodium chloride, sodium citrate, mannitol, and sorbitol to minimize red blood cell lysing and to enhance filtration.

The filtered sample migrates from the filtration membrane 103 to the dry detection membrane 102. For example, the blood serum may migrate to the dry detection membrane 102 after separation of the plasma from the red blood cells for analyte detection. The dry detection membrane 102 may then detect the target analyte present in the filtered sample based on fluorescence induced on the dry detection membrane 102. If plasma or blood serum is used as the bodily fluid sample then the filtration membrane 103 may not be required and the dry detection membrane 102 may receive the plasma or the blood serum directly from the sample receptacle 105.

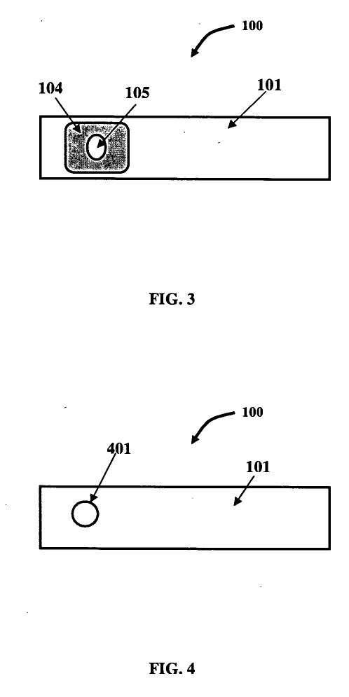

A circular opening 401 on the support platform 101 as illustrated in FIG. 4 may be made. The circular opening 401 may be used as an aperture for enabling a hand held fluorometer to read and measure the emitted fluorescent signals from the dry detection membrane 102. The fluorometer quantifies the measurable properties of the target analyte based on the emitted fluorescent signals. The dry fluorescence biosensor strip 100 may detect and quantify the target analyte in about 10 seconds from time of receiving the bodily fluid sample. The induction of fluorescence on the dry detection membrane 102 results in emission of fluorescent signals from the dry detection membrane 102.

### EXAMPLE 1

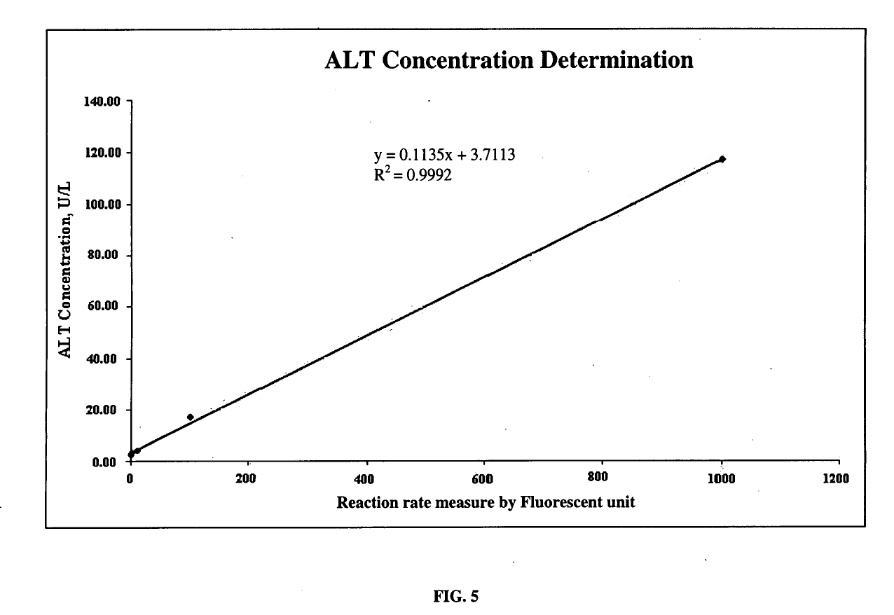

A dry fluorescence biosensor strip 100 of example 1 demonstrates high sensitivity and broad range of the dry fluorescence biosensor strip 100 for detection and quantification of ALT. A reagent solution comprising 100 mM of potassium phosphate buffer with a pH of 7.4, 700 mM of L-alanine, 0.1% by volume of alpha-ketoglutaric acid, 10 mM of magnesium chloride, 0.01% by volume of thiamine pyrophosphate acid, 5 mM of ethylene diamine tetraacetic acid, 0.2% by volume of gelatin, 18 units/mL of horseradish peroxidase and pyruvate oxidase each, and 0.005% by volume of ADHP is prepared. Biodyne along with a detection membrane is dipped in the reagent solution and excess liquid is blotted off with a glass rod. The impregnated detection membrane is then dried completely in circulating air, at 25° C. temperature and less than 30% relative humidity, and used as the dry detection membrane 102. The dry detection membrane 102 is then used in the dry fluorescence biosensor strip 100 as illustrated in FIG. 1, using SG membrane as filtration membrane 103. Serially diluted ALT standard samples of quantities 0 units/L, 1 units/L, 10 units/L, 100 units/L, and 1000 units/L are prepared in 10% bovine serum albumin. The ALT concentrations are measured by applying 10 microliters of one of the serially diluted ALT standard samples onto a dry fluorescence biosensor strip 100 and the emitted fluorescent signal is read and measured by a handheld fluorometer. A graph correlating ALT concentration with the reaction rate measured by fluorescent intensity for serially diluted ALT standard samples is illustrated in FIG. 5.

### EXAMPLE 2

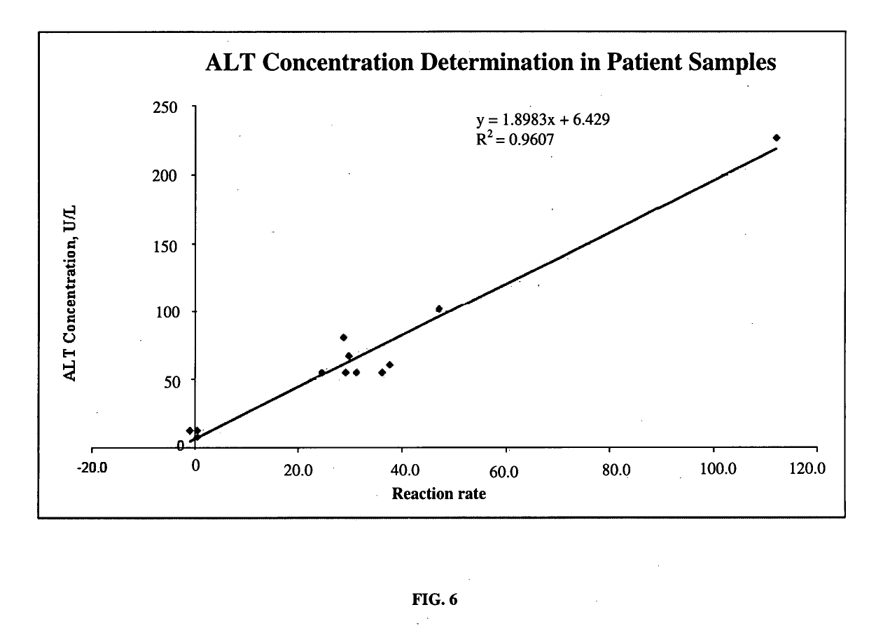

A dry fluorescence biosensor strip 100 of example 2 determines ALT concentrations in patient serum. A group of patient bodily fluid samples are tested with the dry fluorescence biosensor strip 100 for ALT detection and accurate correlation is observed. A graph correlating ALT concentration with the reaction rate measured by fluorescent intensity for patient bodily fluid samples is illustrated in FIG. 6.

### EXAMPLE 3

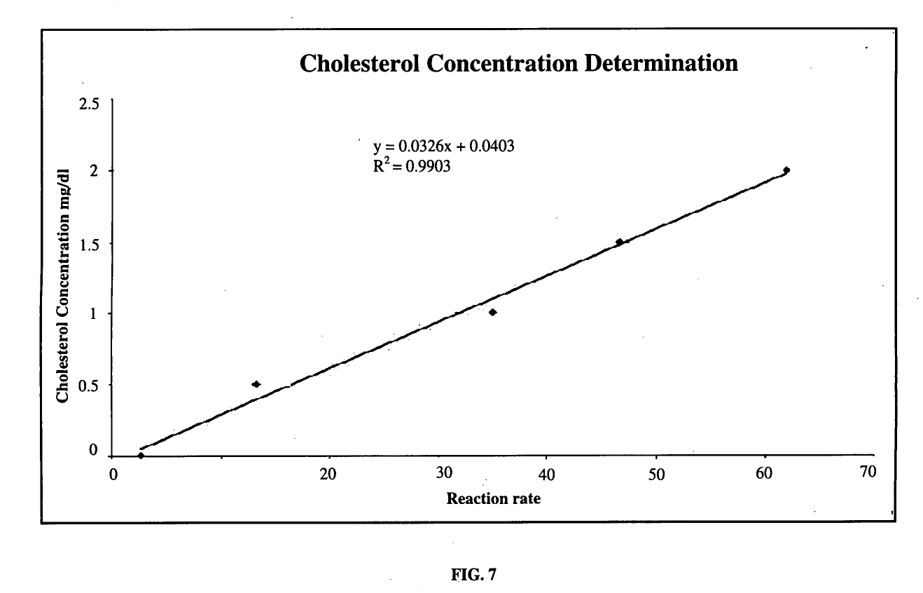

A dry fluorescence biosensor strip 100 of example 3 demonstrates high sensitivity of the dry fluorescence biosensor strip 100 for cholesterol detection. A reagent solution is prepared comprising 50 mM potassium phosphate buffer with a pH of 7.4, 25 mM sodium chloride, 0.1% by volume of cholic acid, 1% by volume Triton X-100, 0.2% by volume gelatin, 100 units/mL of horseradish peroxidase, 100 units/mL of cholesterol esterase, 100 units/mL of cholesterol oxidase, and 0.005% by volume of ADHP. Biodyne along with a membrane is dipped in the reagent solution and excess liquid is blotted off with a glass rod. The impregnated membrane is dried completely in circulating air, at 25° C. temperature and less than 30% relative humidity, and used as the dry detection membrane 102. The dry detection membrane 102 is then used in the dry fluorescence biosensor strip 100 as illustrated in FIG. 3, with SG membrane as the filtration membrane 103. Serially diluted free cholesterol standard samples of quantities 0 mg/dL, 0.5 mg/dL, 1.0 mg/dL, 1.5 mg/dL, and 2.0 mg/dL are prepared in 10% bovine serum albumin. Cholesterol concentrations are measured by applying 10 microliters of one of the serially diluted free cholesterol standard samples onto the dry fluorescence biosensor strip 100. The emitted fluorescent signals are read and measured by a handheld fluorometer. A graph correlating cholesterol concentration with the reaction rate measured by fluorescent intensity is illustrated in FIG. 7.

The foregoing examples have been provided merely for the purpose of explanation and are in no way to be construed as limiting of the present invention disclosed herein. While the invention has been described with reference to various embodiments, it is understood that the words, which have been used herein, are words of description and illustration, rather than words of limitation. Further, although the invention has been described herein with reference to particular means, materials and embodiments, the invention is not intended to be limited to the particulars disclosed herein; rather, the invention extends to all functionally equivalent structures, methods and uses, such as are within the scope of the appended claims. Those skilled in the art, having the benefit of the teachings of this specification, may effect numerous modifications thereto and changes may be made without departing from the scope and spirit of the invention in its aspects.

I claim:

1. A dry fluorescence bio sensor strip for rapid detection of a target analyte present in bodily fluids, comprising:
a sample receptacle for receiving a sample of one of said bodily fluids; and
to a dry detection membrane impregnated with fluorogenic substrates and enzymes, wherein said dry detection membrane detects presence of said target analyte in said received sample based on fluorescence induced on the dry detection membrane;
whereby a reaction between the target analyte, said fluorogenic substrates, and said enzymes induces said fluorescence rapidly on the dry detection membrane, thereby enabling said rapid detection of the target analyte present in the bodily fluids.

2. The dry fluorescence biosensor strip of claim 1, further comprising a filtration membrane for filtering the received sample, wherein said filtered sample migrates from said filtration membrane to the dry detection membrane.

3. The dry fluorescence biosensor strip of claim 2, further comprising an adhesive patch for binding the filtration membrane and the dry detection membrane on a support platform.

4. The dry fluorescence biosensor strip of claim 1, wherein fluorescent signals are emitted from the dry detection membrane on said induction of the fluorescence.

5. The dry fluorescence biosensor strip of claim 4, wherein said emitted fluorescent signals from the dry detection membrane are read and measured by a fluoro meter.

6. The dry fluorescence biosensor strip of claim 1, wherein the dry detection membrane detects presence of alanine aminotransferase in the bodily fluids.

7. The dry fluorescence biosensor strip of claim 1, wherein the dry detection membrane detects presence of aspartic acid aminotransferase in the bodily fluids.

8. The dry fluorescence biosensor strip of claim 1, wherein the fluorogenic substrates are selected from a group comprising dihydrofluoresceins, dihydrocalcein, dihydrorhodamines, dihydroethidium, and 10-acetyl-3,7-dihydroxyphenoxazine.

9. The dry fluorescence biosensor strip of claim 1, wherein said enzymes comprise a fluorescence causing enzyme and analyte specific enzymes.

10. The dry fluorescence biosensor strip of claim 9, wherein said fluorescence causing enzyme is horseradish peroxidase.

11. The dry fluorescence biosensor strip of claim 1, wherein the target analyte is one of glucose, cholesterol, triglyceride, uric acid, and creatine kinase.

12. The dry fluorescence biosensor strip of claim 1, wherein said bodily fluid sample is one of blood, blood serum, plasma, saliva, urine, mucous fluid, milk, and urea.

13. The dry fluorescence biosensor strip of claim 1, wherein the target analytes are detected in about ten seconds from time of receiving said bodily fluid sample.

14. A method of rapid detection of a target analyte present in bodily fluids, comprising the steps of:
providing a dry fluorescence biosensor strip, comprising:
a sample receptacle for receiving a sample of one of said bodily fluids; and
a dry detection membrane impregnated with fluorogenic substrates and enzymes, wherein said dry detection membrane detects presence of said target analyte in said received sample from said sample receptacle;
introducing said bodily fluid sample into the sample receptacle of the dry fluorescence biosensor strip; and
detecting presence of the target analyte in said introduced sample using the dry fluorescence biosensor strip based on fluorescence induced on the dry detection membrane;
whereby a reaction between the target analyte, said fluorogenic substrates, and said enzymes induces said fluorescence rapidly on the dry detection membrane, thereby enabling said rapid detection of the target analyte present in the bodily fluids.

15. The method of claim 14, wherein fluorescent signals are emitted from the dry detection membrane on said induction of the fluorescence.

16. The method of claim 15, further comprising a step of quantifying measurable properties of the target analyte based on said emitted fluorescent signals utilizing a fluorometer.

17. The method of claim 14, wherein the fluorogenic substrates are selected from a group comprising dihydrofluoresceins, dihydrocalcein, dihydrorhodamines, dihydroethidium, and 10-acetyl-3,7-dihydroxyphenoxazine.

18. The method of claim 14, wherein said enzymes comprise a fluorescence causing enzyme and analyte specific enzymes, wherein said fluorescence causing enzyme is horseradish peroxidase.

19. The method of claim 14, wherein the dry fluorescence biosensor strip further comprises a filtration membrane for filtering the received sample from the sample receptacle, wherein said filtered sample migrates from said filtration membrane to the dry detection membrane.

20. The method of claim 14, wherein the target analyte is one of glucose, cholesterol, alanine aminotransferase, aspartic acid aminotransferase, triglyceride, uric acid, and creatine kinase.

21. The method of claim 14, wherein the bodily fluid sample is one of blood, blood serum, plasma, saliva, urine, mucous fluid, milk, and urea.

22. The method of claim 14, wherein the target analyte is detected and quantified in about ten seconds from time of introduction of the bodily fluid sample on the dry fluorescence biosensor strip.

23. A method of preparing a dry detection membrane for detecting presence of alanine aminotransferase in bodily fluids, comprising the steps of:
preparing a reagent solution comprising about 100 mM of potassium phosphate buffer with a pH of about 7.4, about 700 mM of L-alanine, about 0.1% by volume of alpha ketoglutaric acid, about 10 mM magnesium chloride, about 0.01% by volume of thiamine pyrophosphate acid, about 5 mM of ethylene diamine tetraacetic acid, 0.2% by volume of gelatin, about 18 units/mL of horseradish peroxidase, about 18 units/mL of pyruvate oxidase, and about 0.005% by volume of 10-Acetyl-3,7-dihydroxyphenoxazine;
impregnating a detection membrane with said reagent solution; and
drying said impregnated detection membrane in circulating air at a temperature of about 25° C. and relative humidity less than about 30%, wherein said dried detection membrane detects said presence of said alanine aminotransferase in said bodily fluids.

24. The method of claim 23, wherein the dry detection membrane is utilized in a dry fluorescence biosensor strip to detect presence of the alanine aminotransferase in the bodily fluids using a fluorometer.

25. The method of claim 23, wherein the dry detection membrane is utilized in a dry fluorescence biosensor strip to detect presence of aspartic acid aminotransferase in the bodily fluids using a fluorometer.
